(19) United States
(12) Patent Application Publication
(10) Pub. No.: US 2011/0028962 A1
(43) Pub. Date: Feb. 3, 2011

(54) ADJUSTABLE PULMONARY VEIN ABLATION CATHETER

(76) Inventors: Randell WERNETH, San Diego, CA (US); Timothy J. CORVI, Carlsbad, CA (US); Ricardo ROMAN, San Diego, CA (US)

Correspondence Address: Christopher & Weisberg, P.A., 200 East Las Olas Boulevard, Suite 2040, Fort Lauderdale, FL, 33301, US

(21) Appl. No.: 12/533,361

(22) Filed: Jul. 31, 2009

(51) Int. Cl. A61B 18/18; A61B 18/14

(52) U.S. Cl. 606/33; 606/41

(57) ABSTRACT

A medical device is provided, including an elongate body defining a lumen therethrough; a shaft extending through the lumen; and an electrode array coupled to the elongate body at a first end and coupled to the shaft at a second end, where linear manipulation of the shaft causes the electrode array to transition from a first geometric configuration to a second configuration, and where rotational manipulation of the shaft causes the electrode array to transition from the second geometric configuration to a third configuration.

### CROSS-REFERENCE TO RELATED APPLICATION

n/a

### STATEMENT REGARDING FEDERALLY SPONSORED RESEARCH OR DEVELOPMENT

n/a

### FIELD OF THE INVENTION

The present invention relates to a medical method and system having a selectively configurable treatment array, and a method and system for modifying and controlling the shape and/or dimensions of a treatment array.

### BACKGROUND OF THE INVENTION

Numerous procedures involving catheters and other minimally invasive devices may be performed to provide a wide variety of treatments, such as ablation, angioplasty, dilation or the like. For example, to treat cardiac arrhythmias, physicians often employ specialized ablation catheters to gain access into interior regions of the body. Such catheters include tip electrodes or other ablating elements used to create ablation lesions that physiologically alter the ablated tissue without removal thereof, and thereby disrupt and/or block electrical pathways through the targeted tissue. In the treatment of cardiac arrhythmias, a specific area of cardiac tissue having aberrant electrically conductive pathways, such as atrial rotors, emitting or conducting erratic electrical impulses, is initially localized. A user (e.g., a physician) directs a catheter through a main vein or artery into the interior region of the heart that is to be treated. Subsequently, the ablating portion of the selected device is next placed near the targeted cardiac tissue that is to be ablated, such as a pulmonary vein ostium or atrium.

An ablation procedure may involve creating a series of inter-connecting lesions in order to electrically isolate tissue believed to be the source of an arrhythmia. During the course of such a procedure, a physician may employ several different catheters having variations in the geometry and/or dimensions of the ablative element in order to produce the desired ablation pattern. Multiple devices having varying dimensions and/or shapes may also be employed to account for variations in anatomical dimensions from patient to patient. Each catheter may have a unique geometry for creating a specific lesion pattern or size, with the multiple catheters being sequentially removed and replaced to create the desired multiple lesions. Exchanging these various catheters during a procedure can cause inaccuracies or movement in the placement and location of the distal tip with respect to the tissue to be ablated, and may further add to the time required to perform the desired treatment. These potential inaccuracies and extended duration of the particular procedure increase the risk to the patient undergoing treatment.

In light of the above, it is desirable to provide a medical device having multiple, controllable shapes or dimensions, thereby reducing or eliminating the need for additional medical devices having varied, but limited geometric orientations, and thus, limited ability to provide multiple ablative patterns or sizes.

### SUMMARY OF THE INVENTION

The present invention advantageously provides a medical device providing multiple controllable shapes or dimensions, thereby reducing or eliminating the need for additional medical devices having varied, but limited geometric orientations, and thus, limited ability to provide multiple ablative patterns or sizes.

In particular, a medical device is provided including an elongate body defining a lumen therethrough; a shaft extending through the lumen; and an electrode array coupled to the elongate body at a first end and coupled to the shaft at a second end, where linear manipulation of the shaft causes the electrode array to transition from a first geometric configuration to a second configuration, and where rotational manipulation of the shaft causes the electrode array to transition from the second geometric configuration to a third configuration. The medical device may include a linear actuator coupled to the shaft for the linear manipulation thereof or a rotational actuator coupled to the shaft for the rotational manipulation thereof. The electrode array may include a plurality of electrodes, where at least one of the plurality of electrodes defines an asymmetrical cross section. The first geometric configuration can be a substantially linear configuration, the second geometric configuration can include one of a helical or circular configuration defining a first diameter, and the third geometric configuration can include one of a helical or circular configuration defining a second diameter either greater than the first diameter or less than the first diameter (depending on the direction of rotational manipulation).

An intravascular catheter is also provided, having a catheter body defining a proximal portion and a distal portion; a shaft extending from the distal portion of the catheter body; a carrier arm coupled to the catheter body; a distal tip defining a first lumen and a second lumen, where a portion of the shaft is disposed within the first lumen and a portion of the carrier arm is disposed within the second lumen; and an electrode array disposed on the carrier arm. The catheter may include a handle assembly coupled to the proximal portion of the catheter body, and the handle assembly may include a linear actuator coupled to the shaft for the longitudinal movement thereof, where the linear actuator element is releasably securable in a plurality of discrete positions on the handle assembly, or a rotational actuator coupled to the shaft for the rotation thereof, where the rotational actuator element is releasably securable in a plurality of discrete positions on the handle assembly.

A method for ablating a tissue region is provided, including positioning a treatment assembly of a medical device proximate a tissue region, the treatment element containing an electrode array having a first end coupled to a catheter body, and a second end coupled to a shaft extending from the catheter body; manipulating the shaft in a linear direction to controllably transition the plurality of electrodes from a first geometric configuration to a second geometric configuration; manipulating the shaft in a rotational direction to controllably transition the plurality of electrodes from the second geometric configuration to a third geometric configuration; and delivering ablative energy to the treatment assembly. The method may include manipulating the shaft in a first rotational direction to obtain a third geometric configuration defining a second diameter greater than the first diameter, and manipulating the shaft in a second rotational direction to obtain a third geometric configuration defining a second diameter less than the first diameter. The method may also include monitoring an electrical signal of the tissue region, such as a cardiac tissue region.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is an illustration of an embodiment of a medical system constructed in accordance with principles of the present invention;

FIG. 2 is an illustration of an embodiment of a medical device constructed in accordance with principles of the present invention;

FIG. 3 is an illustration of an embodiment of a treatment assembly in a first geometric configuration in accordance with principles of the present invention;

FIG. 4 is an illustration of an embodiment of a treatment assembly in a second geometric configuration in accordance with principles of the present invention;

FIG. 5 is an illustration of an embodiment of a treatment assembly in a third geometric configuration in accordance with principles of the present invention;

FIG. 6 is an illustration of an embodiment of a treatment assembly in an alternative third geometric configuration in accordance with principles of the present invention;

FIG. 7 is an illustration of an embodiment of a distal tip of a medical device constructed in accordance with principles of the present invention;

FIG. 8 is an illustration of an embodiment of an electrode constructed in accordance with principles of the present invention;

FIG. 9 is an illustration of another embodiment of an electrode constructed in accordance with principles of the present invention; and

FIG. 10 is an illustration of an embodiment of a handle assembly of a medical device constructed in accordance with principles of the present invention.

### DETAILED DESCRIPTION OF THE INVENTION

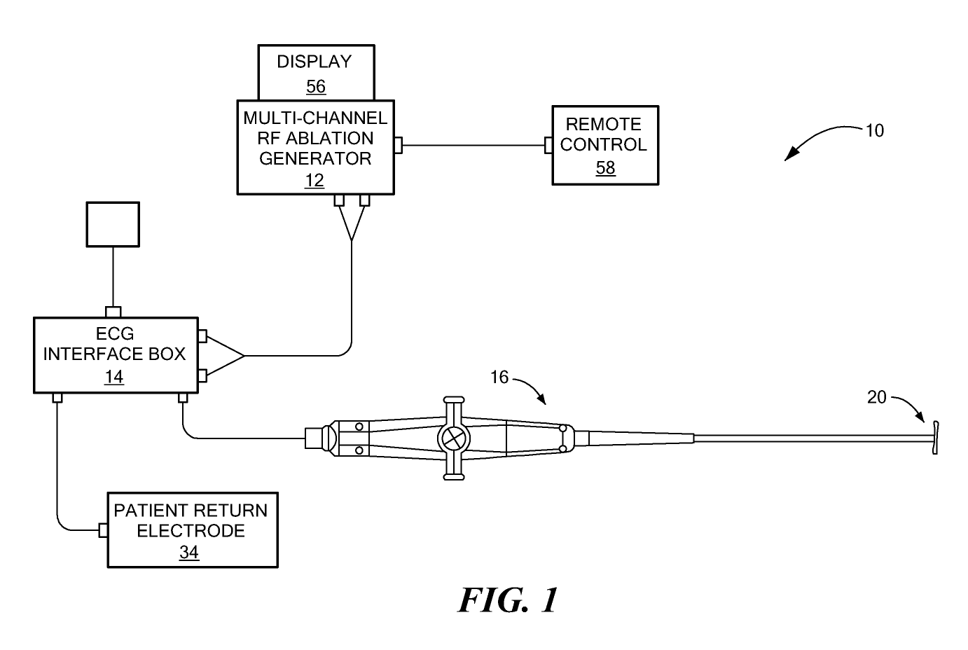

The present invention advantageously provides a medical system and components thereof providing multiple controllable shapes or dimensions that can be selectively manipulated to provide varying treatment patterns. In particular and as shown in FIG. 1, an ablation therapy system, generally designated as ‘10,’ is provided for treating unwanted tissue conditions, including atrial fibrillation or other arrhythmias. The ablation therapy system may generally include a radiofrequency (“RF”) signal generator 12 having a user interface for the operation and control thereof, an electrocardiogram (“ECG”) unit 14 operably coupled to or otherwise interfaced with the RF signal generator 12, and a medical device 16 operably coupled to or otherwise interfaced with the RF signal generator 12 and/or the ECG unit 14.

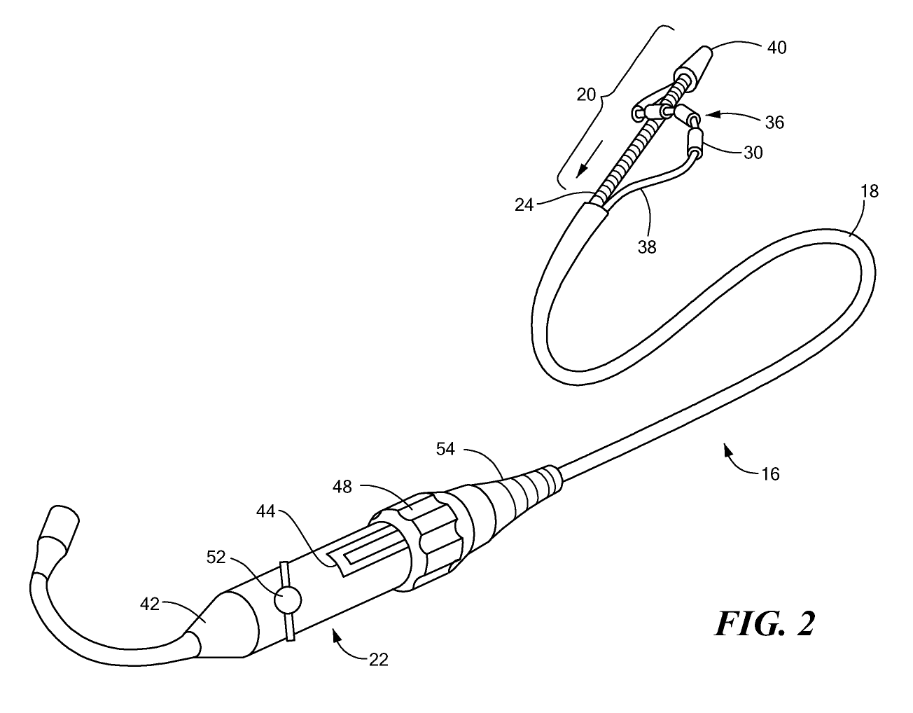

Now referring to FIGS. 1-2, the medical device 16 may include a catheter sized and dimensioned to intraluminally and transseptally access a left atrium of a patient's heart for the subsequent treatment or ablation thereof. The medical device 16 may generally define an elongated, flexible catheter body 18 having a distal treatment assembly 20, as well as a handle assembly 22 at a proximal end or portion of the catheter body 18. The catheter body 18 may define a lumen that slideably receives a shaft 24 therethrough, and may be formed and dimensioned to provide sufficient column and torsional strength to support standard interventional procedures such as those which access the vasculature from a femoral vein or artery and further access the patient's heart. The shaft 24 may define one or more lumens 26 therethrough, to allow for the passage of a guidewire or the like therethrough. The shaft 24 and/or the catheter body 18 may include reinforcement elements or otherwise be constructed to provide desired degrees of stiffness, flexibility, and/or torque transmission along the length of the body and at discrete locations along the length thereof. For example, the catheter body 18 may include wires, braiding, increased wall-thickness, additional wall layering, sleeves, or other components reinforcing or otherwise supplementing an outer wall or thickness along its length. Discrete portions that may experience significant loading or torque during a particular procedure may also include such reinforcement.

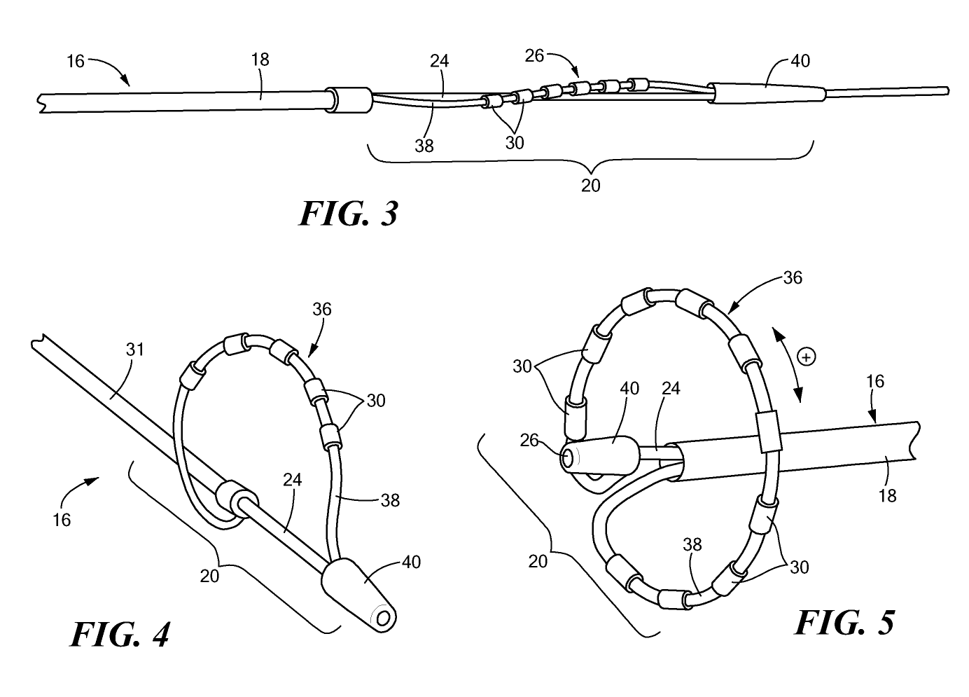

Now referring to FIGS. 3-6, the distal treatment assembly 20 provides for the treatment, monitoring, and/or otherwise clinically interacting with a desired tissue region, such as the heart. The treatment assembly 20 may include, for example, an electrode array 28 disposed near, on, or substantially on the distal end of the catheter body. The electrode array 28 may include a plurality of electrodes 30 along its length. These electrodes 30 may be mounted to detect electrical signals between any pair of electrodes (bi-pole) for mapping of electrical activity, and/or for performing other functions such as pacing of the heart. Moreover, the electrodes 30 may deliver ablation energy across an electrode pair or from independent electrodes when delivering monopolar energy. In a particular example, the plurality of electrodes may include from four (4) to sixteen (16) electrodes with symmetric or asymmetric spacing. Each electrode 30 may include an integral thermocouple (not shown) located on or near the tissue side of the electrode to monitor the temperature at each ablation site before and during ablation. The electrodes 30 may be constructed from platinum, iridium, gold, silver or the like, and may measure approximately about 3 mm in length and separated by a distance of approximately 1 mm to approximately 4 mm, for example.

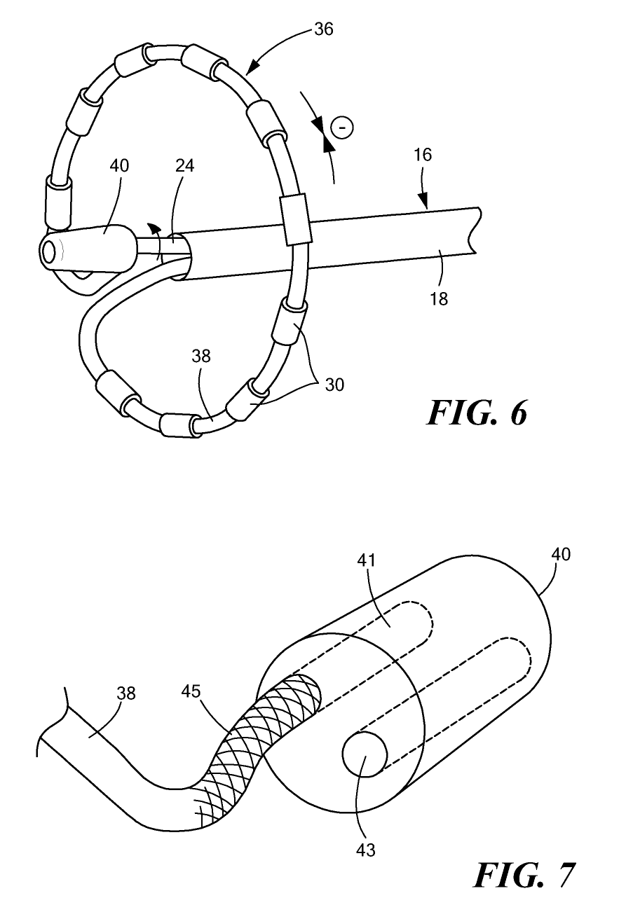

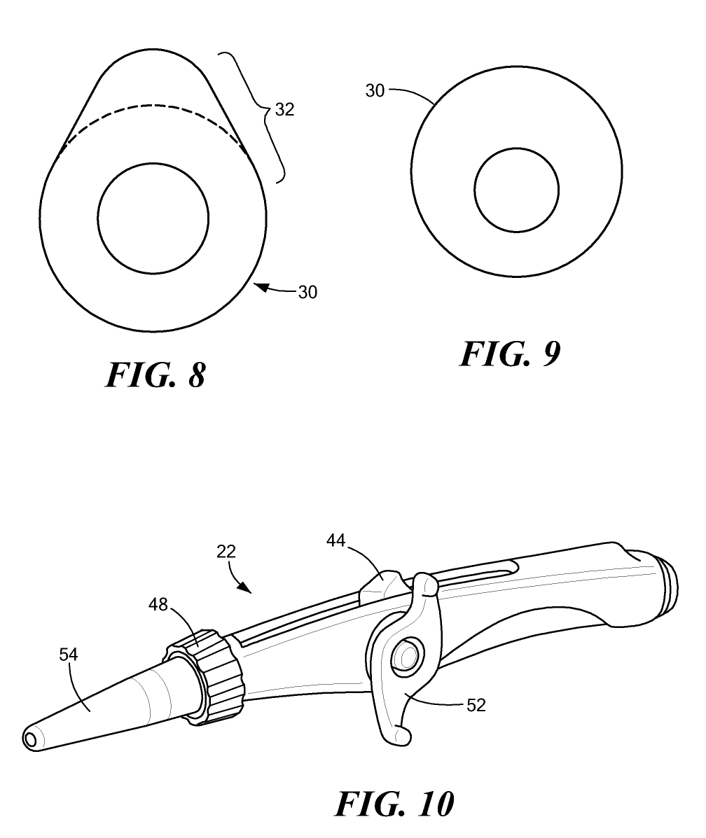

Each electrode 30 may further define an asymmetrical cross section or otherwise provide an increased surface area to increase cooling of the electrodes. During an ablative procedure, for example, a portion of one or more electrodes may contact tissue to deliver therapeutic thermal treatment (via radiofrequency, for example). While each electrode may annularly circumscribe a portion of the medical device, the surface or portion of the electrode facing away from the contacted tissue may be exposed to blood flow (or other fluid flow in the case of an irrigated surgical site), which cools the heated electrode. Providing an increased surface area or thermal volume exposed to the cooling effects of the flowing fluid increases the heat dissipation of the electrode 30. Reducing the temperature of the electrode allows increased radiofrequency power output, which subsequently allows for deeper ablative lesions and may further reduce near-field charring of tissue in closest proximity to each electrode. Now referring to FIGS. 6-7, examples of asymmetrical electrodes having increased surface areas opposite a tissue contacting surface are shown. The electrode 30 may include an extension or fin 32 of material on the opposite side (FIG. 8), or may include a larger wall thickness and/or radius on the side opposite tissue contact (FIG. 9). The electrodes 30 may further be angularly oriented or positioned about the carrier arm 38 to increase the fluid flow patterns about the electrodes 30 when the medical device 10 is in operation.

Each of the electrodes may be electrically coupled to the RF signal generator 12, which may also be attached to a patch electrode 34, such as a conductive pad attached to the back of the patient, to enable the delivery of monopolar ablation energy when desired. While monopolar and bipolar RF ablation energy may be the selected forms of energy to pass through the electrodes of the medical device, other forms of ablation energy may be additionally or alternatively emitted from the treatment assembly, including electrical energy, magnetic energy, microwave energy, thermal energy (including heat and cryogenic energy) and combinations thereof. Moreover, other forms of energy that may be applied can include acoustic energy, sound energy, chemical energy, photonic energy, mechanical energy, physical energy, radiation energy and a combination thereof.

The electrode array 28 may be arranged in a resiliently biased manner and have specific geometric configurations which generally allow them to ablate specific tissue (such as a pulmonary vein, for example) having predetermined or otherwise known geometric or topographical characteristics. The electrode array 28 may be selectively movable from a primary, stored or delivery configuration for transport and delivery to the treatment site (such as a radially constrained configuration) to multiple secondary, deployed or expanded configurations for treatment.

As shown in FIGS. 3-6, the treatment assembly 20 of the medical device 16 may include a carrier assembly 36 that supports the electrode array 28 thereon. The carrier assembly 36 may include a flexible carrier arm 38 having one end coupled to the catheter body 18 and/or handle assembly 22, and an opposite end coupled to a distal tip 40. As shown in FIG. 7, the distal tip 40 may define a first lumen 41 for receiving and/or coupling to a portion of the carrier arm 38, and a second lumen 43 for coupling and/or receipt of a portion of the shaft 24 therein. The distal tip 40 may be constructed form an electrically conductive material and used for mapping, pacing, ablating or otherwise electrically interacting with a targeted tissue region. The distal tip 40 may further define a through-hole into the lumen extending proximally through the shaft 24. The lumen may extend and terminate at a guidewire exit 42 on the handle assembly 22. As such, the catheter body 18 and distal treatment assembly 20 may be percutaneously advanced over a guidewire, such as a guidewire inserted into a pulmonary vein of the patient. The carrier arm 38 may be constructed from a shape memory material, such as nitinol, to provide one or more pre-determined and/or biased geometric configurations. Conventional marking elements (e.g. radiopaque markers) may be included in the distal treatment assembly, carrier assemblies or other components of the medical device to determine the relative location of the carrier assembly and/or the deployment condition of the carrier assembly, as well as confirm contact with tissue.

As the carrier assembly 36 is coupled to the distal end of the shaft 24 by the distal tip 40, the shaft 24 can be manipulated to control the geometry of the carrier assembly 36 and thus the electrode array 28. For example, the shaft 24 can be retracted to transition the carrier arm 38 from a near linear configuration (as shown in FIG. 3) to a partial circumferential (less than 360.degree.) loop (i.e., a partial helical or spiral shape, as shown in FIG. 4). Advancement and/or retraction of the shaft 24 can adjust the geometry of the loop of the electrode array 28, such as increasing/decreasing the diameter of the carrier arm 38. Moreover, rotation of the shaft 24 can also increase and decrease the diameter of the carrier arm 38, and thus the electrode array 28. FIG. 5 shows an increased diameter for the electrode array resulting from rotation of the shaft 24 in a first direction, while FIG. 6 shows decreased diameter for the electrode array resulting from rotation of the shaft 24 in a second direction Rotating the shaft 24 imparts a torque and/or rotation to the distal tip 40. As the carrier assembly is also coupled to the distal tip 40, the torque and/or rotation delivered by the shaft 24 is transferred to the carrier assembly, thereby causing the carrier assembly 36, and thus the electrode array 28, to increase or decrease in diameter, depending upon which direction the shaft is turned. In one example, the range of usable diameters of the carrier assembly 36 may range from about 15 mm to a diameter of about 35 mm to accommodate varied anatomical contours neighboring pulmonary vein ostia (including non-circular ostia) or other vascular features.

The carrier assembly 36 may include reinforcement elements or otherwise be constructed to provide desired degrees of stiffness, flexibility, and/or torque transmission along its length or at discrete locations along the length thereof. For example, the carrier arm 38 may include wires, braiding, increased wall-thickness, additional wall layering, sleeves, or other components reinforcing or otherwise supplementing an outer wall or thickness at the junction or region in proximity to the distal tip 40 to minimize the likelihood of structural failure resulting from the experienced torque or strain transmitted from the shaft 24 through the distal tip 40. Moreover, the dual-lumen construct of the distal tip 40 may provide improved torsional transmission from the shaft 24 to the carrier arm 38 while maintaining the structural integrity of both the shaft 24 and the carrier arm 38 where they couple to the distal tip 40.

The handle assembly 22 of the medical device may include one or more mechanisms or components to facilitate manipulation of the shaft and/or the distal treatment assembly. For example, as shown in FIG. 10, the handle assembly 22 may include a linear actuator 44 providing for the proximal-distal extension and retraction of the shaft 24. The linear actuator 44 may be movably coupled to a portion of the handle assembly 22 to allow it to slide or otherwise translate in a proximal-to-distal direction, and vice versa. The handle assembly 22 may further include a housing 46 coupled to the linear actuator 44 and/or handle assembly 22 to facilitate movement and/or linkage of the actuator and the shaft 24.

A rotational actuator 48 may also be disposed on or about the handle assembly 22 to facilitate rotation of the shaft 24 (and thus the distal treatment assembly 20, including the carrier assembly 36 and electrode array 28, as described above) about a longitudinal axis of the catheter body 18 in two directions. As described above, the geometric configuration (e.g., the radius, dimensions, shape) of the electrode array 28 may be manipulated and controlled through manipulation of the shaft 24. The rotational actuator 48 may be directly coupled to the shaft, or alternatively, include one or more intermediary components to effectuate a controllable, mechanical linkage between the rotational actuator and the shaft, such as a secondary gear assembly.

One or more internal push/pull wires may also be provided in the medical device, and in particular, coupled to the handle assembly. For example, to facilitate single or bi-directional steering and control of the distal treatment assembly, a full length pull wire (or double pull wires such as in the case with bi-directional steering, neither of which is shown) may be secured to the a distal portion of the end of the shaft 24. The pull wire may extend proximally to a steering knob 52. Rotation of the knob 52 may pull the wire that, in turn, controls the plane in which the electrodes contact tissue.

The medical device may further include a capture element 54 that is friction fit over a distal end of the handle assembly 22. The capture element 54 may be configured to be detached therefrom and slide in a distal direction over the catheter body 18 until the electrode array 28 is received therein, in a stored or confined configuration. The capture element 54 may be applied over the electrode array 28 for constraint and protection thereof during delivery through a hemostasis valve of a transseptal sheath or a vascular introducer. In this manner, the array may be introduced safely (e.g. without damage) into the patient's vasculature (e.g., a femoral vein). After introduction of electrode array 28 through the hemostasis valve, the capture element 54 may be moved proximally over the catheter body and reattached to the distal end portion of the handle assembly 22 to function as a strain relief.

The RF signal generator 12 functions to generate RF energy as supplied to selected catheter electrodes or between selected pairs of electrodes for the electrode array, to ablate or otherwise treat cardiac tissue. In particular, the RF signal generator 12 may be configured to generate and control the delivery of RF energy based on temperature feedback from the respective thermocouple of each electrode. Each electrode 30 may be independently monitored followed by temperature-controlled delivery of RF energy. Energy delivery may further automatically be duty-cycled to maximize the delivery of RF energy to the electrode based on the measured tissue temperature. Hence, as the tissue temperature increases due to delivery of RF energy (resistive heating), the electrodes 30 in turn increase in temperature, as monitored by the corresponding thermocouple. For instance, during bipolar delivery, if the set target temperature of the electrodes is 60° C. and one of the two electrodes is monitored at 55° C., while the other electrode is monitored to be at 50° C., the generator will selectively limit energy delivery based on the needs of one electrode measured at 55° C. This prevents either electrode of the pair from ever significantly surpassing the set target temperature. In contrast, during a monopolar phase of the energy delivery, the RF signal generator will deliver RF energy to each electrode 30 solely based on the temperature measured by its corresponding thermocouple. The temperature measurements may be performed between RF duty cycles (off-cycles) to minimize interference and to optimize accuracy of temperature readings.

The RF signal generator 12 may also include a user interface 56 and/or a remote control 58 (shown in FIG. 1). The user interface 56 allows a user to select parameters for the desired mapping and/or ablation treatment. The user interface 56 may allow the user to select an energy delivery mode for the treatment. For example, the user interface 56 can allow the user to select the delivery of only monopolar energy, only bipolar energy, or a combination of the two. The user interface may also allow the user to select a power ratio, such as 1:1, 2:1, or 4:1, when in combination mode. The generator 12 can be manufactured to include specific alternative power ratios (e.g., 1:1, 2:1, 4:1), such that the user can select one of the established ratios, and/or the user interface can allow the user to enter a different power ratio. The user interface 56 may also allow the user to change the energy mode when the catheter is changed, or when the medical device is moved to a different location in order to ablate different tissue.

The ECG unit 14 is provided to monitor and map signals detected by the electrodes of each electrode array. These two units (i.e., the RF signal generator 12 the ECG unit 14) may be interfaced in parallel, via the ECG interface 14, to the medical device 16. The ECG unit 14 electrically isolates the ECG unit 14 from any damaging signals generated by the RF generator 12. Any RF energy signals reaching the ECG unit 14, especially signals of the magnitude generated by the RF generator 12, would likely damage the monitor unit's amplifiers. ECG unit 14 may also be configured to isolate the ECG monitoring unit from electrical noise generated by the delivery of the RF energy.

In an exemplary use of the present system, the medical device 16 may be used to investigate and treat aberrant electrical impulses or signals in a selected tissue region, such as in the heart. Primarily, the distal treatment assembly 20 may be advanced through the patient's vasculature via the femoral vein over a previously inserted guidewire. The distal treatment assembly 20 may then be advanced into the right atrium and into proximity of a pulmonary vein, for example. In order to advance the carrier assembly 36 through the vasculature and into the desired position, the distal treatment assembly 20 (including the carrier assembly 36 and the electrode array 28) may be oriented in a first, substantially linear transport configuration (FIG. 3). The first, substantially linear transport configuration may be achieved through the manipulation of the linear actuator 44 on the handle assembly 22 (by extending the shaft 24 to a distal-most point, for example). In turn the flexible carrier arm 38 may be urged toward the substantially linear configuration. In this linear orientation, the carrier assembly is minimized and compact in a transverse dimension for easily advanced through the vasculature (or a trans septal sheath).

Once in the desired proximity to the target tissue, the carrier assembly 36 and the electrode array 28 may be deployed into a second, expanded geometric configuration (FIG. 4). To achieve the second geometric configuration, the linear actuator 44 may be retraced in a proximal direction to thereby retract the shaft 24 and the distal tip 40 into a proximal direction as well. Thus, while the longitudinal length of the carrier assembly 36 is decreasing, the radial dimension of the deploying electrode array 28 can be increasing. The carrier assembly 36 can be further advanced towards the target tissue while simultaneously retracting the shaft 24 (via the linear actuator) to deploy the electrode array 28.

Once the resiliently biased carrier arm 38 of the carrier assembly 36 has extended radially into a partial helical or spiral configuration, the radius of the electrode array 28 can be selectively and controllably increased or decreased into a third geometric configuration (FIGS. 5-6) by rotating or torqueing a portion of the shaft 24. The manipulation of the shaft 24 to impart a subsequent change in the dimensions and radius of the electrode array 28 can be facilitated by operation of the rotational actuator 48 on the handle assembly 22. As previously indicated, by rotation of the control shaft 24 about its longitudinal axis, the shape and diameter of the electrode array loop can be adjusted. This permits accommodation of the various anatomical contours, such as that neighboring the pulmonary vein ostia (including non-circular ostia), as well as enabling the operator to adjust the size and shape of the array to best suit the particular patient's anatomy (including the pulmonary vein ostia, for example). The operational diameter of the carrier assembly 36 may be configured between a diameter of about 10 mm to about 50 mm.

Upon obtaining the desired geometric configuration of the carrier assembly and electrode array, the steering mechanism of the medical device (e.g., the steering knob 52 and the internal pull wire or wires) may be used to deflect the distal tip to contact the target tissue At this juncture, the geometric configuration of the electrode array 28 can be further adjusted to achieve optimal contact with the surrounding targeted tissue. By way of example, adjusting the rotational actuator 48 in a counterclockwise direction (when viewed from a proximal-to-distal direction) may increase the diameter of the electrode array 28, while adjusting the rotational actuator 48 in the clockwise direction decreases the diameter of the electrode array 28, or vice versa. The selective, controllable expansion and/or restriction of the electrode array diameter provides increased accuracy and greater range for placement of the electrode array in proximity to the precise location and tissue desired.

Sufficient contact with tissue may be determined when the carrier assembly transitions to a convex shape or through fluoroscopic imaging. In addition, the location and tissue contact can be confirmed using the electrodes 30 of the medical device. For example, an electrophysiologist can map the contacted tissue to not only determine whether or not to ablate any tissue, but to also confirm tissue contact which is identified in the mapping procedure. If conditions are determined to be inadequate, an operator may adjust the shape of carrier assembly (e.g. through advancement or retraction of shaft 24, or rotation of the rotational actuator 48 to impart larger or smaller diameters) and/or the operator may reposition carrier assembly 36 against tissue through various manipulations performed at the proximal end of medical device. Moreover, it will be appreciated that other conventional mapping catheters can be applied to map signals, such as a standard electrophysiology lasso catheter.

Once sufficient tissue contact has been established and the mapping procedure has confirmed the presence of aberrant conductive pathways, ablation energy may be passed through the electrodes 30 (i.e., 5-10 Watts) of the electrode array 28. The electrode array 28 and the RF signal generator 12 may cooperate to deliver RF energy in monopolar, bipolar or combination monopolar-bipolar energy delivery modes, simultaneously or sequentially, and with or without durations of terminated energy delivery.

Depending upon a number of primary factors, such as the geometry and location of targeted tissue region, the quality of the electrode/tissue contact, the selected magnitude of the RF energy delivered to the electrodes, the type of RF energy applied, as well as the duration of the ablation, lesion formation can be estimated that is sufficient to eliminate aberrant conductive pathways therethrough. For example, given the above factors, a target temperature of the ablated tissue may be about 60° C., with a lower limit of about 55° C. and an upper limit of about 65° C.

The ability to selectively change the dimensions of the electrode array 28 allows a single medical device to accommodate anatomical differences experienced from one patient to another (e.g., one patient may require a particular treatment element radius, while a different patient may require an increased treatment element radius), while also providing a single device with the ability to provide multiple ablation treatment patterns or sizes (e.g., elongated, annular, etc.) within a single patient. The need for multiple devices having differing, but fixed, shapes or sizes is reduced or eliminated altogether.

While examples and illustrations of particular geometric configurations have been provided, it is understood that virtually any shapes, configurations, and/or dimensions may be included and/or achieved by the treatment array of the medical device of the present invention, including but not limited to those shapes illustrated and described herein. A particular geometric configuration may include circular, conical, concave, convex, rounded, or flattened features and/or combinations thereof. Accordingly, an embodiment of the medical device of the present invention may be able to provide focal lesions, circular lesions, linear lesions, circumferential lesions, and combinations thereof having varying dimensions.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A medical device, comprising:
an elongate body defining a lumen therethrough;
a shaft extending through the lumen; and
an electrode array coupled to the elongate body at a first end and coupled to the shaft at a second end, wherein linear manipulation of the shaft causes the electrode array to transition from a first geometric configuration to a second configuration, and wherein rotational manipulation of the shaft causes the electrode array to transition from the second geometric configuration to a third configuration.

2. The medical device according to claim 1, further comprising a linear actuator coupled to the shaft for the linear manipulation thereof.

3. The medical device according to claim 2, further comprising a rotational actuator coupled to the shaft for the rotational manipulation thereof.

4. The medical device according to claim 1, wherein the electrode array includes a plurality of electrodes, and wherein at least one of the plurality of electrodes defines an asymmetrical cross section.

5. The medical device according to claim 1, wherein the first geometric configuration is a substantially linear configuration, the second geometric configuration includes one of a helical or circular configuration defining a first diameter, and the third geometric configuration includes one of a helical or circular configuration defining a second diameter greater than the first diameter.

6. The medical device according to claim 1, wherein the first geometric configuration is a substantially linear configuration, the second geometric configuration includes one of a helical or circular configuration defining a first diameter, and the third geometric configuration includes one of a helical or circular configuration defining a second diameter less than the first diameter.

7. The medical device according to claim 1, further comprising:
an electrocardiograph unit in electrical communication with the electrode array; and
a radiofrequency signal generator in electrical communication with the electrode array.

8. An intravascular catheter, comprising:
a catheter body defining a proximal portion and a distal portion;
a shaft extending from the distal portion of the catheter body;
a carrier arm coupled to the catheter body;
a distal tip defining a first lumen and a second lumen, wherein a portion of the shaft is disposed within the first lumen and a portion of the carrier arm is disposed within the second lumen; and
an electrode array disposed on the carrier arm.

9. The intravascular catheter according to claim 8, further comprising a handle assembly coupled to the proximal portion of the catheter body.

10. The intravascular catheter according to claim 9, wherein the handle assembly includes a linear actuator coupled to the shaft for the longitudinal movement thereof.

11. The intravascular catheter according to claim 10, wherein the linear actuator element is releasably securable in a plurality of discrete positions on the handle assembly.

12. The intravascular catheter according to claim 9, wherein the handle assembly includes a rotational actuator coupled to the shaft for the rotation thereof.

13. The intravascular catheter according to claim 12, wherein the rotational actuator element is releasably securable in a plurality of discrete positions on the handle assembly.

14. The intravascular catheter according to claim 8, wherein the electrode array includes a plurality of electrodes, and wherein at least one of the plurality of electrodes defines an asymmetrical cross section.

15. A method for ablating a tissue region, comprising:
positioning a treatment assembly of a medical device proximate a tissue region, the treatment element containing an electrode array having a first end coupled to a catheter body, and a second end coupled to a shaft extending from the catheter body;
manipulating the shaft in a linear direction to controllably transition the plurality of electrodes from a first geometric configuration to a second geometric configuration;
manipulating the shaft in a rotational direction to controllably transition the plurality of electrodes from the second geometric configuration to a third geometric configuration; and
delivering ablative energy to the treatment assembly.

16. The method according to claim 15, wherein the first geometric configuration is a substantially linear configuration.

17. The method according to claim 16, wherein the second geometric configuration includes one of a helical or circular configuration defining a first diameter.

18. The method according to claim 17, wherein the third geometric configuration includes one of a helical or circular configuration defining a second diameter greater than the first diameter.

19. The method according to claim 17, wherein the third geometric configuration includes one of a helical or circular configuration defining a second diameter less than the first diameter.

20. The method according to claim 17, wherein manipulating the shaft in a rotational direction to controllably transition the plurality of electrodes from the second geometric configuration to a third geometric configuration includes manipulating the shaft in a first rotational direction to obtain a third geometric configuration defining a second diameter greater than the first diameter, and manipulating the shaft in a second rotational direction to obtain a third geometric configuration defining a second diameter less than the first diameter.

21. The method according to claim 15, wherein the tissue region includes cardiac tissue.
